(19) United States
(12) Patent Application Publication
(10) Pub. No.: US 2018/0256176 A1
(43) Pub. Date: Sep. 13, 2018

(54) MEDICAL ASPIRATION SYSTEM AND MEDICAL THROMBECTOMY SYSTEM

(71) Applicant: Acandis GmbH & Co. KG, Pfinztal (DE)

(72) Inventor: Giorgio Cattaneo, Baden-Württemberg (DE)

(21) Appl. No.: 15/754,895

(22) PCT Filed: Aug. 24, 2016
(86) PCT No.: PCT/EP2016/069965

§ 371 Date: Feb. 23, 2018

(30) Foreign Application Priority Data
Aug. 25, 2015 (DE) 10 2015 114 044.6

(51) Int. Cl. A61B 17/22; A61F 7/12

(52) U.S. Cl. CPC A61B 17/22; A61F 2007/0063; A61B 2017/22079; A61F 7/12

(57) ABSTRACT

The invention relates to a medical aspiration system having a catheter with at least two lumens, wherein a first lumen forms an aspiration lumen which is connected to an aspiration unit for suction removal of substances from a blood vessel through an inlet opening in the aspiration lumen, and a second lumen forms an infusion lumen through which a substance can be supplied to the blood vessel through an outlet opening in the infusion lumen, wherein the outlet opening in the infusion lumen is arranged in a region between the inlet opening of the aspiration lumen and a position proximal to the inlet opening in the aspiration lumen. The invention is characterized in that the infusion lumen is connected to a cooling unit, which supplies a cooled liquid for administration through the infusion lumen.

The invention relates to a medical aspiration system having a catheter with at least at least two lumens, a first lumen forming an aspiration lumen which is connected to an aspiration unit for suction removal of substances from a blood vessel through an inlet opening in the aspiration lumen. A second lumen forms an infusion lumen through which a substance can be supplied to the blood vessel through an outlet opening in the infusion lumen. The outlet opening of the infusion lumen is arranged in an area between the inlet opening of the aspiration lumen and a position proximal to the inlet opening in the aspiration lumen.

Such an aspiration system is known from U.S. Pat. No. 5,554,137, for example.

The known aspiration system is used to administer medications and to remove thrombi by aspiration. To do so the aspiration system has a catheter with two working lumens which are closed at the distal end and have slotted valves for aspiration and infusion. The problem with such catheters is that because of the slotted valves they have a relatively complicated design and are designed only for aspiration in combination with administration of medications.

U.S. Pat. No. 5,554,137 proposes that the catheter be provided with a special profile and a certain stiffness in the area of the slotted valves. However this chances about the fact that such aspiration systems are complicated.

The object of the invention is to improve upon an aspiration system of the type defined in the introduction in view of the complexity and gentle treatment.

According to the invention, this object is achieved by an aspiration system having the features of claim 1. Alternatively, this object is achieved by a medical aspiration system having the features of the other independent claim 10. Further alternatively, the object is achieved by a medical aspiration system having the features of the other independent claim 12 and by a medical thrombectomy system having the features of the other independent claim 13.

The invention is based on the idea of providing a medical aspiration system which has a catheter with at least two lumens, wherein a first lumen forms an aspiration lumen which is connected to an aspiration unit for suction removal of substances from a blood vessel. The suction removal of the substances takes place through an inlet opening in the aspiration lumen. A second lumen forms an infusion lumen through which a substance can be supplied to the blood vessel. The supply takes place through an outlet opening in the infusion lumen. The outlet opening in the infusion lumen is arranged in an area between one of either the inlet opening of the aspiration lumen and a position proximal to the inlet opening of the aspiration lumen. The infusion lumen is connected to a cooling unit, in particular with a fluid connection which supplies a cooled fluid for administration through the infusion lumen.

The inlet opening in the aspiration lumen and the outlet opening in the infusion linen are each situated in the distal region of the catheter, i.e., on the blood side during use. The term inlet opening relates to the aspiration function and the entrance of the substances drawn into the aspiration lumen. The term “outlet opening” relates to the infusion function and the emergence of the liquid supplied from the infusion lumen.

The invention has the advantage that the connection of the infusion lumen to a cooling unit permits a particularly gentle means of removing thrombi from the blood circulation due to the protective effect of the hypothermia treatment. Therefore the cooling unit supplies a liquid at a temperature lower than body temperature. This liquid is administered through the infusion lumen connected to the cooling unit. This achieves a cooling of the treated blood vessels in a simple and effective manner. The cooling can take place simultaneously with the aspiration so that possible complications caused by the aspiration are reduced. The invention has the advantage that this achieves an effective cooling without any additional cooling elements. According to the invention, the cooled liquid is dispensed directly into the blood vessel through the infusion lumen. Therefore, the infusion lumen is advantageously open at the distal end. In any case the cooling unit forms part of the aspiration system according to the invention.

This eliminates the complicated design of the catheter from the prior art. Instead, the invention permits the use of a multi-lumen catheter, i.e., a catheter having at least two lumens, wherein at least the aspiration lumen, in particular the aspiration lumen and the infusion lumen are open at the distal end of the catheter. The thrombus can therefore be sucked in through the aspiration lumen or introduced together with a recanalization unit and/or a thrombectomy unit into the aspiration lumen. The thrombus is then in the aspiration lumen and can be removed.

In contrast with an arrangement in which the infusion lumen ends distally from the aspiration lumen, the invention arrangement of the outlet opening and the inlet opening in conjunction with the cooling unit has the advantage that the cooled liquid emerging from the infusion lumen is not drawn through the aspiration lumen or at least is drawn through it only to a reduced extent. Therefore an effective cooling is achieved.

This is the case in particular when branches in the vessel or collateral vessels are situated at the level of the outlet of the infusion lumen or between the outlet of the infusion lumen and the inlet of the aspiration lumen. It is thus possible to supply cold infusion [solution] to the collateral vessels during the aspiration. This arrangement is important in all phases of the procedure even if there is no aspiration. For example, the aspiration lumen can be used to supply fluid from distal catheters, for example, aspiration catheters and intermediate catheters and systems for mechanical thrombectomy. During the administration, the aspiration lumen is fleshed with infusion solution at a low flow rate so that the supply takes place smoothly. In this phase it is advantageous if the cold infusion solution passes through a separate lumen which is not blocked partially by such a catheter or other thrombectomy systems.

In this way higher flow rates of cold infusion solution are possible than if the cold infusion were to take place in the same lumen.

The infusion lumen differs from the aspiration lumen in its diameter. The aspiration lumen has a larger diameter than the infusion lumen. In addition the aspiration lumen preferably has a catheter wall which has greater dimensional stability than the catheter wall of the infusion lumen. This prevents the catheter from collapsing during aspiration. Since an excess pressure prevails in the infusion lumen when liquids are being supplied, the problem of collapse of the infusion lumen occurs only to a minor extent or not at all.

The catheter wall of the aspiration lumen in particular can be reinforced, for example, by a metal and/or some other additional plastic material. A friction-reducing inner layer may be provided. Thus the supply of additional catheters, aspiration catheters and intermediate catheters or systems for the thrombectomy is facilitated. The infusion lumen can be reinforced by a metal and/or by some other plastic material. The variant without metal, i.e., with only an additional material, is to be preferred for the infusion lumen because the lumen is not inclined to collapse because of the small dimensions. The two lumens can both be reinforced by another material. Alternatively only one lumen is reinforced and the other lumen is not reinforced. The aspiration lumen optimally has at least one metal reinforcement and a friction-reducing plastic layer. The infusion lumen has an additional reinforcing material. The infusion lumen is preferably embedded in the same plastic material in which the aspiration lumen is also embedded. It may be in direct contact with the plastic (i.e., no other material is present for reinforcement) or it has a reinforcing material. It may also be that the same material reinforcement encloses both lumens at the same time. In this case, an additional plastic material is provided, which encloses the metal reinforcement. The interior plastic has two lumens, for example, made of PTFE or some other material. The aspiration lumen may have an inner layer of Teflon.

According to the other independent claim 12, an aspiration lumen which is connected to an aspiration lumen of the catheter may be provided. Furthermore, the infusion lumen of the catheter may be connected to an infusion device which provides a liquid, in particular a cooled liquid for administration through the infusion lumen. Otherwise, claim 12 corresponds to the subject matter of claim 1.

According to the other independent claim 13, a medical cooling system comprising a catheter with an instrument lumen may be provided. The instrument lumen may serve to feed a medical instrument into a blood vessel. Otherwise, claim 13 corresponds essentially to the subject matter of claim 1.

The medical cooling system is preferably part of a set. The set comprises the medical cooling system and a medical instrument which is or can be positioned so that it is longitudinally displaceable in the instrument lumen. The catheter may be arranged directly inside the instrument lumen. Alternatively, a microcatheter may also be passed through the instrument lumen which holds the instrument.

For all the embodiments of the invention, it holds that an additional (micro)catheter can basically be inserted into the aspiration lumen and/or the instrument lumen, wherein said additional (micro)catheter is aspirated distally or accommodates the thrombus with the help of self-expanding devices used for mechanical thrombectomy. This microcatheter is then retracted into the aspiration lumen and/or the instrument lumen while being sucked into the aspiration lumen and/or instrument lumen or not. A thrombus can be removed or retracted through the aspiration lumen and/or instrument lumen regardless of whether the retraction is accomplished by means of a vacuum, for example, through a syringe or through another aspiration device, or purely mechanically through the mechanical thrombectomy, among other means. It is also possible for multiple (micro)catheters to be arranged in a “telescopic” formation. Only one of the (micro)catheters or all the (micro) catheters may have an infusion lumen for administration of cold fluid.

Preferred embodiments of the invention are the subject matter of the dependent claims.

The aspiration lumen and the infusion lumen are preferably separated in a fluid-tight manner by wall without any valves. The catheter may be designed as a simple continuous tube having a multiple lumen. Between the individual multiple lumens there are no connections, which simplifies production of the catheter. Furthermore, the fluid-tight separation of the lumens prevents any pieces of thrombi that might be in the aspiration lumen during administration of the infusion solution from being flushed out of the catheter.

In particular the infusion can be continued without being influenced by removal of the thrombus, even after conclusion of thrombectomy. Additional checks on the condition of the vessel are possible, for example, by administering contrast agents through the aspiration lumen while the infusion being administered through the infusion lumen.

Production of the catheter is simplified if the aspiration lumen and the infusion lumen are designed in one piece.

Alternatively, the at least two lumens of catheter may be arranged so that they are longitudinally displaceable into one another. The lumens may in particular be positioned coaxially one inside the other and longitudinally displaceable relative to one another. In concrete terms, it is possible to provide that the aspiration lumen or instrument lumen and/or the function lumen is/are arranged coaxially inside the infusion lumen.

It should be emphasized that the term catheter is also understood to include “airlocks.” Airlocks are also positioned in the vessel directly by means of mandrels, also known as “dilators” without using any additional outer tubing. The use as an airlock is particularly advantageous because the dimensions of an airlock may be selected to larger than those in a catheter. The use of a larger lumen is thus conceivable for both aspiration and infusion.

In a preferred embodiment, the area between the inlet opening of the aspiration lumen and the position proximally to the inlet opening of the aspiration lumen amounts to at least 0 cm, in particular at least 5 cm, in particular at least 10 cm, in particular at least 20 cm. The larger the distance in the outlet opening of the infusion lumen from the inlet opening of the aspiration lumen, the smaller the amount of cooled liquid that is drawn into the aspiration lumen and can flow in vascular branches without being aspirated. The distance from the outlet opening of the infusion lumen to the inlet opening of the aspiration lumen should not be more than 30 cm, so that the efficacy of the cooling in the area of the inlet opening of the aspiration lumen is retained.

The length of the catheter may be at least 80 cm, in particular at least 90 cm (position in the ACC). The catheter may alternatively have a length of at least 90 cm, at least 100 cm, at least 110 cm (position in the ACI, MCA). The diameter of the catheter may be at least 7 Fr, in particular at least 8 Fr and at most 11 Fr, in particular at most 10 Fr, in particular at most 9 Fr (position in the ACC). Alternatively, the catheter may have a diameter between 5 and 7 Fr, preferably 6 Fr (position in the ACI, MCA).

If the catheter is considered to be an “airlock,” the aspiration lumen may be compatible with a catheter having a diameter of at least 4 Fr, 5 Fr or 6 Fr. The upper limit is 6 Fr. In the optimum case, the outside diameter should be max. 13 Fr, in particular max. 12 Fr, in particular max. 11 Fr as the airlock.

The cross-sectional area of the infusion lumen may be between 0.16 mm² and 0.4 mm². Thus, a volume flow great enough for cooling can be conveyed through the infusion lumen.

For applications in which the cold liquid is administered in a very short period of time, the lumen may have a diameter between 0.16 mm² and 1 mm², in particular between 0.2 mm² and 0.8 mm², in particular between 0.3 mm² and 0.6 mm².

When the cooling unit and the infusion lumen are releasably connected, the cooling unit can be replaced at least temporarily by a different function unit, wherein the cooling unit remains part of the aspiration system. This embodiment has a supportive effect to facilitate the removal of the thrombus. For example, the size of the thrombus can be reduced or it can be comminuted or made softer due to a supply of lysis medium. The function unit may comprise a feed unit filled with lysis medium or contrast medium, said feed unit being connected to the infusion lumen. Here again it is true that a simultaneous treatment of a patient during an aspiration is also possible. The removal of thrombi by aspiration is supported by the lysis medium. The use of contrast medium facilitates the positioning of the aspiration lumen based on the thrombus to be removed. After aspiration, an angiographic test can be performed to test the patency of the vessel and optionally initiate additional steps. For example, one step may consist of removing the thrombus with a self-expanding device after aspiration has been incomplete or failed or, if a self-expanding device has already been used, guiding it to the thrombus one additional time (iterative recanalization process).

The invention is additionally based on the idea of providing a medical aspiration system with a catheter having a single lumen. The single lumen is connected to two separate function units by a switchable valve. In a first switch position of the valve, a first function unit is fluidically connected to the single lumen and in a second switch position of the valve a second function unit is fluidically connected to the single lumen. The first function unit has an aspiration unit for suction removal of substances from a blood vessel, and the second function unit has a cooling unit for supplying cooled liquid to the blood vessel. Therefore a cold infusion medium can be infused prior to the removal of the thrombus. For aspiration of the thrombus, the valve is switched accordingly so that the thrombus can be removed with suction. After removing the thrombus, the valve is switched again so that a cold infusion solution can be administered as needed.

The medical aspiration system with the catheter which has a single lumen may be part of a set. The set preferably comprises the aspiration system with the catheter and a medical instrument which is or can be positioned in the lumen, where it is longitudinally displaceable. The instrument may be guided directly or by means of a microcatheter through the lumen.

The inventive aspiration system has a particularly simple design because of the single lumen catheter and fulfills various functions at the same time by switching the valve, without any complex changing of different function units.

Another aspect of the invention relates to a set having a medical cooling system which has a catheter with a single lumen which is connected to a cooling unit for supplying cooled liquid to a blood vessel and which has a valve proximally to an outlet opening in the lumen, in particular a hemostatic valve. A function catheter for supplying a medical instrument or for aspiration is or can be positioned in the lumen so that it is longitudinally displaceable, so that an annular space for supplying the cooled liquid remains between the catheter and the function catheter.

The valve may form a branch, in particular in the form of a Y, wherein a first leg of the branch is fluidically connected to the cooling unit and a second leg of the branch is adapted for insertion of the function catheter into the single lumen. A closure means, in particular a hemostatic valve membrane, may be arranged inside the second leg, retaining the liquid but being puncturable by the function catheter. Cooled liquid and a function catheter may be guided into a blood vessel at the same time in this way.

The function catheter may have a greater length than the catheter. For example, the function catheter may be longer than the catheter and/or the lumen of the catheter by at least 10%, in particular at least 20%, in particular at least 30%. The function catheter may in particular protrude distally beyond the outlet opening of the lumen of the catheter during use, so it can extend further than the catheter into the blood vessel. Medical instruments can be supplied through the function catheter or suction (aspiration) may take place through the function catheter.

The outlet opening of the lumen is preferably arranged in an area between a distal inlet opening of the function catheter and a position proximally to the inlet opening of the lumen. Cooled liquid can thus be directed into a blood vessel through the lumen of the catheter in a targeted manner, so that the treatment area at the tip of the function catheter is cooled well. The cooled liquid then flows through the annular gap between the catheter and the function catheter.

In general, it holds for the present patent application that medical instruments can be supplied into a blood vessel through the catheter and/or through the instrument catheter. In particular, stents, coils, flow diverters, thrombectomy devices, additional catheters or microcatheters as well as medical substances such as adhesives are regarded here as medical instruments.

In both embodiments the invention is not limited to the aspiration of thrombi or to a thrombectomy. In treatment of stenoses in the heart in the periphery or intracranially in particular, through a balloon or through a stent, it may be advantageous for small particles of plaque to be aspirated and/or for the plaque dilatation to be carried out with aspiration. In parallel, it is advantageous to administer cold liquid to induce a neuroprotective effect in the case of distal vascular occlusions due to particles of plaque being flushed out. Here again, the aspiration lumen may be used in the broadest sense, i.e., without aspiration, e.g., for feeding a stent into position. In addition, it is possible to treat spasms with this catheter. Here again, the aspiration lumen may be used to advance a catheter through the spasm and possibly also to induce dilatation of the spasm by means of a balloon. Cold liquid can be administered for cooling in parallel through the infusion lumen. The catheter can also be used for treatment of aneurysms and malformations. Here again, the cooling has a neuroprotective effect in the event of hemorrhaging while the aspiration lumen makes it possible to feed catheters into position for treatment, e.g., through coils, stents, flow diverters and/or adhesives.

The invention is explained in greater detail below on the basis of schematic drawings with additional details, in which:

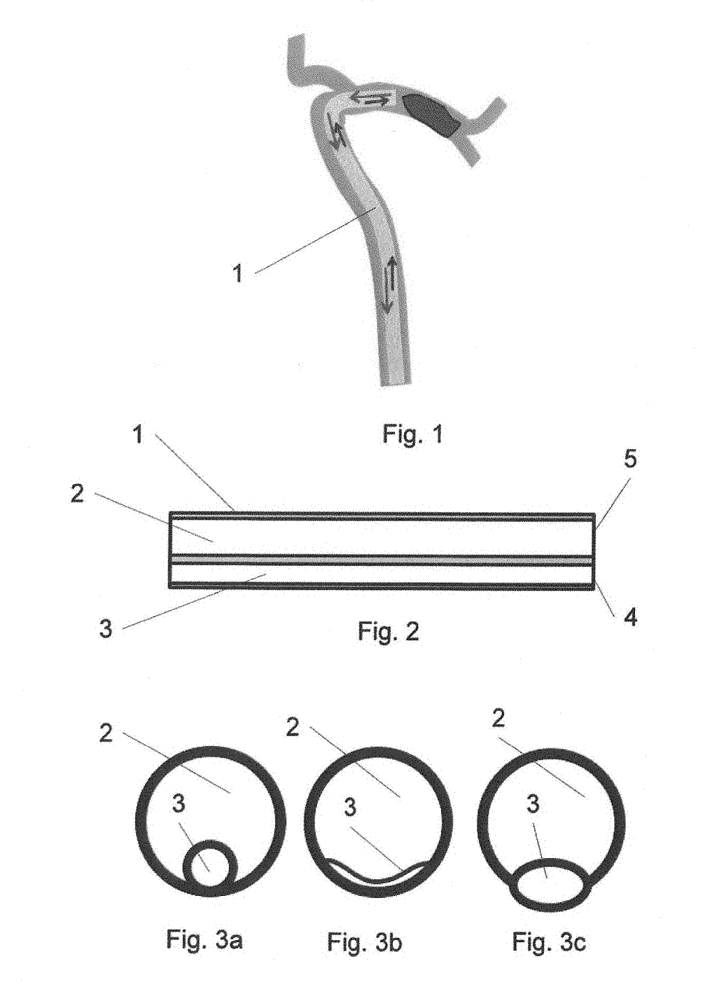

FIG. 1 shows a two-lumen catheter in a blood vessel upstream from a thrombus in which aspiration is being performed and a transfusion is being administered at the same time;

FIG. 2 shows a longitudinal section through the catheter according to FIG. 1;

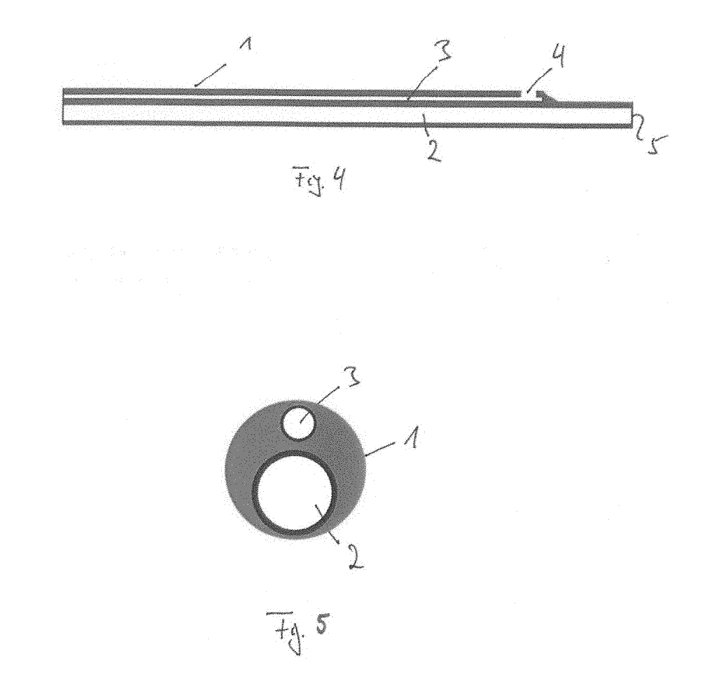

FIGS. 3a, 3b, 3c show a cross section through various embodiments of a two-lumen catheter suitable for the invention;

FIG. 4 shows a longitudinal section through a catheter according to another inventive exemplary embodiment; and

FIG. 5 shows a cross section through a catheter according to another inventive exemplary embodiment.

FIG. 1 shows the catheter 1 of a medical aspiration system according to an inventive exemplary embodiment during treatment. The catheter 1 is therefore inserted into a blood vessel. The tip of the catheter is situated directly upstream from the thrombus to be removed from the blood vessel. The catheter 1 has two lumens. The first lumen forms an aspiration lumen 2 which is connected to an aspiration unit for aspiration of the thrombus (not shown). The aspiration unit may be a suction device or a syringe. The second lumen is an infusion lumen 3, through which a substance, for example, a cooled liquid, is supplied to the blood vessel. The infusion lumen 3 is connected to a function unit (not shown), which is designed as a cooling unit.

As shown in FIG. 2, the infusion lumen 3 has an outlet opening 4 and the aspiration unit has an inlet opening 5. The outlet opening 4 and the inlet opening 5 are formed in the area of the distal catheter tip for aspiration of the thrombus, which is arranged during use in the blood vessel. In concrete terms the openings 4, 5 open into the tip of the catheter. In the exemplary embodiment according to FIG. 2, the outlet opening 4 and the inlet opening 5 are arranged at the same height. This means that the two openings 4, 5 are essentially arranged in the same plane. This arrangement forms the distal maximal position that can be assumed by the outlet opening 4 of the infusion lumen 3 based on the inlet opening 5 in the aspiration lumen 1 (distance=0 cm). In deviation from the embodiment illustrated in FIG. 2, the outlet opening 4 may be arranged so that it is set back in the proximal direction so that it is a distance away from the inlet opening 5 in the proximal direction. The greater the distance between the inlet opening 5 and the outlet opening 4, the less cooled fluid is removed through the inlet opening 5.

Furthermore, the probability that the cold infusion will flow into side vessels and branches is then greater. The distance between the two openings 4, 5 should be no more than 30 cm in order for the cooling effect to be retained in the area of the inlet opening 5.

The aspiration system allows simultaneous aspiration of the thrombus and cooling of the vessel. This is indicated by the large and small arrows in the catheter. The large arrows pointing in the proximal direction illustrate the suction flow created by the vacuum produced by the aspiration unit. The suction flow results in the thrombus in the blood vessel being sucked into the catheter, specifically into the aspiration lumen 2 of the catheter 1. At the same time a substance is administered to the blood vessel. The substance stream to be administered is illustrated by the smaller arrows pointing in the distal direction. The substance flow is produced by a function unit (not shown) which generates an excess pressure conveying the substance into the blood vessel.

The aspiration and the infusion need not necessarily take place at the same time. The infusion can be interrupted during the aspiration. Even if the infusion and the aspiration do not take place at the same time, the present embodiment has the advantage over the prior art that both phases, i.e., the phase of aspiration and the phase of infusion, can be carried out with one and the same catheter in rapid alternation in time without the need for changing catheters.

Preferably only aspiration is performed through the aspiration lumen. In order to prevent infusion medium from being supplied through the aspiration lumen, the aspiration lumen may be connected only to the aspiration unit. If the aspiration system is handled cautiously accordingly, it is also possible to provide the aspiration lumen with two connections, a first connection being connected to an aspiration unit, and a second connection being connected to an infusion unit. In this case, it is possible to administer contrast medium, for example, through the aspiration lumen, so that cold infusion still flows in the infusion lumen without being influenced. The infusion unit in this case is a syringe, for example. If the infusion takes place only before the aspiration, for example, then there is no risk that pieces of thrombi in the aspiration lumen will be flushed out.

The cooling unit is a cooling system, in which the cooling of the liquid and the creation of the pressure for conveying the liquid take place. Instead of the cooling system, it also possible to use a simple cooled bag, filled with the liquid to be administered.

Instead of a cooling agent, a lysis medium or a contrast medium can also be administered during the aspiration. The supply unit may then be a simple syringe.

FIG. 2 shows a longitudinal section through the catheter according to FIG. 1, wherein the larger diameter of the aspiration lumen 2 is readily apparent in comparison with the infusion lumen 3.

FIGS. 3a, 3b, 3c show different variants of the catheter 1 according to FIG. 1. In the variant according to FIG. 3a, the aspiration lumen 2 and the infusion lumen 3 are arranged eccentrically. This is also true of the variants according to FIGS. 3b, 3c. With the variant according to FIG. 3a, the aspiration lumen 2 and the infusion lumen 3 are each designed with a circular cross section. In variant 3b, the infusion lumen 3 is characterized by a wall that is flexible in some sections. During use, i.e., when the infusion lumen 3 is acted upon with a liquid and/or with pressure, the cross section of the infusion lumen 3 opens up automatically. Alternatively, the wall shown in FIG. 3b with the geometry illustrated there is stable, i.e., it is difficult or impossible to deform.

In the variant according to FIG. 3c the infusion lumen 3 has an oval cross section. The infusion lumen 3 is integrated into the wall of the aspiration lumen 2. It is also possible for the aspiration lumen 2 to be round and for the infusion lumen 3 to be arranged completely outside of the aspiration lumen 2. A sickle-shaped cross section may also be possible here. The outside profile may be round or may have a protrusion.

The drawings illustrate the feature wherein the access opening of the infusion lumen 3 is arranged at least at the same height as the access opening in the larger aspiration volume 2. Alternatively, the access opening in the infusion lumen 3 may be arranged further proximally than the access opening in the aspiration lumen 2.

In this case, the infusion lumen 3 may have a lateral opening and/or an outlet opening 4 as shown in FIG. 4. This may be a simple hole in the outside wall of the catheter. The infusion lumen 3 may be closed in the distal area, for example, by adhesive bonding without altering the profile of the aspiration lumen 2. Alternatively, the aspiration lumen 2 may be larger at the distal end from the opening of the infusion lumen and/or may have a larger cross section because the contour of the infusion lumen 3 (which protrudes inwardly) is no longer present or has a smaller profile. Examples: a two-lumen extrusion and/or a two-lumen tube or at least one extrusion or a corresponding tube having an infusion lumen 3 extends in the proximal region as far as the opening 4 in the infusion lumen 3. Beyond the opening 4, the proximal area is connected to a single lumen extrusion or a corresponding tube or at least one infusion lumen-free extrusion and/or a corresponding tubing. It may happen that the proximal area develops into the distal area. This is possible, for example, when the infusion lumen 3 is pressed outward with a mandrel and with corresponding simultaneous heating. It is then possible for a portion of the profile of the infusion lumen 3 to remain and to protrude inward. It is also possible for the aspiration lumen 2 to remain unchanged and for the outside diameter to be reduced due to the reduction in or disappearance of the infusion lumen 3 distally from the opening in the outside diameter. This is logical in particular in the version with a separate reinforcement of the aspiration lumen 2. In this case the infusion lumen 3 does not protrude inward into the aspiration lumen 2.

Instead the infusion lumen 3 is outside of the reinforcement. In this case, the lumen can be closed by a thermal process, for example, by shrinkage, which would reduce the outside diameter of the catheter. Either the cross-sectional area of the aspiration lumen 2 is increased or the outside diameter of the catheter is reduced distally from the opening in the infusion lumen 2 or both. Alternatively, the two remain unchanged.

The length of the catheter may be at least 70 cm, in particular at least 80 cm, in particular at least 90 cm (position in the ACC, common carotid artery). Alternatively, the catheter may have a length of at least 90 cm, in particular at least 100 cm, in particular at least 110 cm, in particular at least 120 cm, in particular at least 130 cm, in particular at least 140 cm (position in the ACI—internal carotid artery, MCA—middle cerebral artery). The diameter of the catheter may be at least 7 Fr, in particular at least 8 Fr, at most 11 Fr, in particular at most 10 Fr, in particular at most 9 Fr (position in the ACC). Alternatively, the catheter may have a diameter between 5 and 7 Fr, preferably 6 Fr (position in the ACI, MCA).

The inside diameter of the aspiration lumen 2 is approx. 300 μm less than the outside diameter. This refers to the outside diameter of the entire catheter. The wall in the area of the lower limit amounts to 300 μm. In particular the difference is between 200 and 1200 μm, in particular between 300 and 1000 μm, in particular between 400 and 800 μm.

The flow cross section of the aspiration lumen 2 is reduced accordingly by the infusion lumen 3, which protrudes partially or completely into the aspiration lumen 2. With the small dimensions of the catheter 1, in particular the infusion lumen 3, however, the aspiration is not impaired significantly.

Alternatively, the infusion lumen 2 does not protrude into the aspiration lumen 3 as shown in FIG. 5. Instead, both lumens 2, 3 are arranged at the side and in the best case are round. The lumens are enclosed by a round outside profile. In the preferred case, a reinforcement is arranged around each lumen 2, 3. It is possible for the reinforcement to be arranged around only the aspiration lumen 2 or only the infusion lumen 3. A variant without reinforcement is also possible.

The infusion lumen 3 is at most large enough that a catheter of at least 3 Fr, in particular at least a 4 Fr catheter can be transported through the aspiration lumen 2 (for positioning in the ACI/MCA). The remaining aspiration volume 2 in the ACC should be designed for supply of a 5 Fr or 6 Fr catheter. For the posterior cerebral circulation (vertebral artery and basilar artery) the same dimensions are conceivable as those for placement in the ACI.

The cross-sectional area of the infusion lumen 3 may be between 0.16 mm² and 0.4 mm². In particular the lumen may have a diameter between 0.16 mm² and 1 mm², in particular between 0.2 mm² and 0.8 mm², in particular between 0.3 mm² and 0.6 mm².

In general, a combination of the aspiration lumen with balloon cooling is possible. This means that the catheter has more than two lumens, for example, four or five lumens. However in the optimal case the catheter has exactly two lumens. Therefore, the dimensions of the catheter can be miniaturized so that a good aspiration is possible in small intracranial vessels.

The combination of the aspiration system with an occlusion balloon 6 is also possible. The occlusion balloon 6 has good compliance with the vascular wall.

The occlusion balloon 6 may be positioned proximally to both openings 4, 5 when both openings 4, 5 are arranged at the same level at the tip of the catheter. If the opening 4 in the infusion lumen is arranged proximally, then the occlusion balloon 6 may be situated in between. It is therefore possible for the cold infusion not to be aspirated as all during the aspiration and to flow in collateral vessels located proximally. Alternatively, the proximally located opening 4 may also be arranged distally to the occlusion balloon 6 if it is necessary for the cooling to also flow distally during the occlusion.

In deviation from FIG. 2, the infusion lumen 3 can also end further proximally. The aspiration lumen 2 is therefore more flexible at the distal end. A distal reduction in the outside diameter, in particular in the case of the proximal opening in the infusion lumen 3 is also possible. The aspiration lumen 2 may retain the same dimension. In other embodiments with an opening at the same level, a reduction in the outside diameter is also possible.

The aspiration system allows the use of hypertonic solutions as well as cold liquid to reduce the edema. Medications of all types can be administered through the infusion lumen 3. The aspiration unit may have a system with a pressure unit, i.e., a pump or a syringe.

The aspiration lumen 2 may be used to supply a recanalization device and/or a thrombectomy unit. In conjunction with the inventive aspiration system and all the exemplary embodiments of this patent application, therefore, the combination of the catheter 1 with a recanalization device and/or a thrombectomy unit is also disclosed and claimed as an additional exemplary embodiment.

Alternatively, the aspiration lumen 2 may be used to supply a microcatheter with a self-expanding thrombectomy element or a self-expandable recanalization element. In conjunction with the inventive aspiration system and all the exemplary embodiments of this patent application, therefore the combination of the catheter 1 with an additional aspiration catheter is disclosed and claimed as an additional exemplary embodiment, for example, in the form of a set with or without additional self-expanding recanalization device. The additional aspiration catheter may have a size of 5 Fr or 6 Fr or the like. The recanalization device and/or the thrombectomy element may be arranged therein.

1. A medical aspiration system having a catheter with at least two lumens, wherein a first lumen forms an aspiration lumen, which is connected to an aspiration unit for suction removal of substances from a blood vessel through an inlet opening in the aspiration lumen, and a second lumen forms an infusion lumen through which a substance can be supplied to the blood vessel through an outlet opening in the infusion lumen, wherein the outlet opening in the infusion lumen is arranged in a region between the inlet opening of the aspiration lumen and a position proximal to the inlet opening in the aspiration lumen,
characterized in that
the infusion lumen is connected to a cooling unit which supplies a cooled liquid for administration through the infusion lumen.

2. The system according to claim 1,
characterized in that
the aspiration lumen and the infusion lumen are separated in a fluid-tight manner by a wall without valves.

3. The system according to claim 1,
characterized in that
the aspiration lumen and the infusion lumen are designed in one piece.

4. The system according to claim 1,
characterized in that
the region between the inlet opening of the aspiration lumen and the position proximal to the inlet opening in the aspiration lumen amounts to at least 0 cm, in particular at least 5 cm, in particular at least 10 cm, in particular at least 20 cm.

5. The system according to claim 1,
characterized in that
the region between the inlet opening of the aspiration lumen and the position proximal to the inlet opening of the aspiration lumen amounts to at most 30 cm.

6. The system according to claim 1,
characterized in that
the length of the catheter amounts to at least 80 cm, in particular at least 90 cm, in particular at least 100 cm, in particular at least 110 cm.

7. The system according to claim 1,
characterized in that
the diameter of the catheter amounts to at least 7 Fr, in particular at least 8 Fr.

8. The system according to claim 1,
characterized in that
the diameter of the catheter amounts to at most 11 Fr, in particular at most 10 Fr, in particular at most 9 Fr.

9. The system according to claim 1,
characterized in that
the cross-sectional area of the infusion lumen is between 0.16 mm2 and 0.4 mm2, in particular between 0.16 mm2 and 1 mm2, in particular between 0.2 mm2 and 0.8 mm2, in particular between 0.3 mm2 and 0.6 mm2.

10. The medical aspiration system having a catheter with a single lumen, which is connected to at least two separate units by a switchable valve, wherein a first unit is fluidically connected to the lumen in a first switch position of the valve, and a second unit is fluidically connected to the lumen in a second switch position of the valve, wherein the first unit comprises an aspiration unit for suction removal of substances from a blood vessel, and the second unit comprises a cooling unit for supplying cooled liquid into the blood vessel.

11. The set with a system according to claim 10 and a medical instrument, which is or can be positioned longitudinally displaceably inside the lumen.

12. The medical aspiration system having a catheter with at least two lumens, wherein a first lumen forms an aspiration lumen, which is connected to an aspiration unit for suction removal of substances from a blood vessel through an inlet opening in the aspiration lumen, and a second lumen forms an infusion lumen, through which a substance can be supplied to the blood vessel through an outlet opening in the infusion lumen, wherein the outlet opening in the infusion lumen is arranged in a region between the inlet opening of the aspiration lumen and a position proximal to the inlet opening in the aspiration lumen,
characterized in that
the infusion lumen is connected to an infusion device which supplies a liquid, in particular a cooled liquid for administration through the infusion lumen.

13. The medical cooling system having a catheter with at least two lumens, wherein a first lumen forms an instrument lumen for supplying a medical instrument, in particular a thrombectomy device, and a second lumen forms an infusion lumen through which a substance can be supplied to the blood vessel through an outlet opening of the infusion lumen, wherein the outlet opening of the infusion lumen is arranged in a region between the inlet opening of the instrument lumen and a position proximal to the inlet opening of the instrument lumen,
characterized in that
the infusion lumen is connected to a cooling unit which supplies a cooled liquid for administration through the infusion lumen.

14. The set with a system according to claim 13 and a medical instrument, which is or can be positioned longitudinally displaceably inside the instrument lumen.

15. The set with a medical cooling system having a catheter with a single lumen which is connected to a cooling unit for supplying cooled liquid into a blood vessel and has a valve, in particular a hemostatic valve, proximally to an outlet opening in the lumen, wherein a function catheter for supplying a medical instrument or for aspiration is or can be positioned longitudinally displaceably in the lumen such that an annular space for supplying the cooled liquid remains between the catheter and the function catheter.

16. The set according to claim 15,
characterized in that
the function catheter has a greater length than the catheter.

17. The set according to claim 15,
characterized in that
the outlet opening in the lumen is arranged in a region between a distal inlet opening in the function catheter and a position proximal to the inlet opening in the lumen.

18. The system according to claim 13,
characterized in that
the at least two lumens of the catheter are arranged longitudinally displaceably, one inside the other, in particular coaxially one inside the other.

19. The system according to claim 18,
characterized in that
the aspiration lumen or the instrument lumen is arranged coaxially inside the infusion lumen.
